(19) United States
(12) Patent Application Publication
(10) Pub. No.: US 2021/0198180 A1
(43) Pub. Date: Jul. 1, 2021

(54) PROCESS FOR PRODUCING SUBSTITUTED 4-AMINOINDANE DERIVATIVES FROM 2-(HYDROXYALKYL)-ANILINES

(71) Applicant: Bayer Aktiengesellschaft, Leverkusen (DE)

(72) Inventors: Florian ERVER, Wiesbaden (DE); Frank MEMMEL, Guntersblum (DE); Sergii PAZENOK, Solingen (DE)

(21) Appl. No.: 17/056,860

(22) PCT Filed: May 21, 2019
(86) PCT No.: PCT/EP2019/063059

§ 371 Date: Nov. 19, 2020

(30) Foreign Application Priority Data
May 23, 2018 (EP) 18173861.8

(51) Int. Cl. C07C 209/68

(52) U.S. Cl. CPC C07C 209/68; C07C 2602/08

(57) ABSTRACT

The present invention relates to a method for preparing substituted 4-aminoindane derivatives from 2-(hydroxyalkyl)-anilines by cyclization,

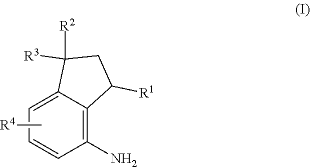

in which the substituents R¹, R², R³ and R⁴ have the definitions as specified in the description.

The present invention relates to a process for preparing substituted 4-aminoindane derivatives by cyclization.

4-Aminoindanes and corresponding derivatives are important intermediates for the preparation of bioactive compounds which can be used specifically for controlling harmful microorganisms in crop protection.

For instance, it is known that various pyrazole indanyl carboxamides have fungicidal activity (e.g. WO 1992/12970, WO 2012/065947, J. Org. Chem. 1995, 60, 1626 and WO 2012/084812).

It is also known that various pyridine indanyl carboxamides have fungicidal activity (e.g. EP-A 0256503, JP-A 1117864, J. Pesticide Sci. 1993, 18, 245).

In addition, it is known that some benzoyl indanyl amides have fungicidal activity (WO 2010/109301).

Very generally, such fungicidal indanyl carboxamides can be produced via the coupling of a 4-aminoindane derivative with an activated heterocyclic acid counterpart by linking the primary amino group of the former with the carboxyl group of the latter (coupling reaction). Concluding, a 4-aminoindane derivative, but also an activated heterocyclic acid that shall be linked to the 4-aminoindane derivative, are important intermediates in the synthesis of fungicidal indanyl carboxamides.

Chemical syntheses of substituted 4-aminoindane derivatives have been described e.g. in WO 2010/109301, WO 2014/103811 and U.S. Pat. No. 5,521,317. However, the described processes only allow the preparation of substituted 4-aminoindanes with very limited substitution patterns. For instance, the methods described in WO 2010/109301 and in WO 2014/103811 only allow the synthesis of an 1,1,3-trimethyl-4-aminoindane derivative starting from aniline by condensation with acetone and exploit the rearrangement reaction described in EP 0654464 and U.S. Pat. No. 5,521,317. U.S. Pat. No. 3,078,319 describes the synthesis of Alkylindanes from olefins using acid catalysts such as sulfuric acid.

A further possibility to prepare 4-aminoindane derivatives is described in WO 2013/167545 and WO 2013/167549. The synthesis is based on a Buchwald-Hartwig amination and thus enables a general synthetic route to substituted 4-aminoindanes. Disadvantages of this method are firstly the cost-intensive use of transition metal catalysts and secondly the problematic synthesis of the corresponding halo-substituted indane precursors. Furthermore, the amino function cannot be introduced directly by free NH₃, but rather requires the use of cost-intensive, protected ammonia derivatives.

Indanes without an amino function on the aromatic ring can be prepared by methods established in classical organic chemistry by Friedel-Crafts cyclizations. To this end, aromatic compounds having hydroxyalkyl or alkene side chains are converted to the corresponding indanes by addition of Brønsted acids such as HCl, HBr, HF, H₂SO₄, H₃PO4, KHSO₄, AcOH, p-toluenesulfonic acid, polyphosphoric acid or of Lewis acids such as AlCl₃, BF₃, AgOTf.

However, it has been shown that, with the exception of polyphosphoric acid, none of the reagents mentioned can be used to prepare 4-aminoindane derivatives by cyclization (J. S. Pizey (Ed.), “Synthetic Reagents 6” Wiley-VCH: New York 1985, 156-414).

In contrast to this, WO 2015/197530 discloses a process for the preparation of 4-aminoindane derivatives which uses as starting materials aromatic compounds having hydroxyalkyl side chains, which can be conducted optionally in the presence of a diluent, which is generally carried out under atmospheric pressure, which is generally carried out at temperatures of from 0° C. to 150° C., preferably at temperatures of from 20° C. to 110° C. and which is carried out in the presence of a suitable Lewis or Bronstedt acid. Listed examples for Lewis acids are metal halides like AlCl₃, BF₃, and other lewis acids known in literature; or triflates, for example silver triflate and other triflates described in the literature. Listed examples for Bronstedt acids are HCl, HBr, HF, H₂SO₄, KHSO₄, AcOH, trifluoroacetic acid, p-toluenesulfonic acid, camphorsulfonic acid, methansulfonic acid, trifluoromethansulfonic acid, polyphosphoric acid, and phosphoric acid. However, according to the preparation examples of WO 2015/197530, the only working combination to yield a 4-aminoindane derivative from the above-mentioned aromatic compounds having hydroxyalkyl side chains is by using polyphosphoric acid as a cyclization mediator at a temperature of 80° C.

However, even this use of polyphosphoric acid is afflicted with disadvantages. On the one hand, for example, the handling of the high-viscosity polyphosphoric acid is extremely inconvenient; on the other hand, an enormous amount of water is required to dissolve and dispose of this after completion of the reaction. In addition, a large amount of unwanted phosphate-containing waste is formed. Finally, the reaction leads only to a moderate yield of 52% of the product 3-ethyl-1,1-dimethylindan-4-amine.

Surprisingly, WO 2017/133981 discloses that substituted 4-aminoindane derivatives can be prepared from aromatic compounds having hydroxyalkyl side chains which are converted to the corresponding 4-aminoindane derivatives by addition of sulfonic acids. In detail, WO 2017/133981 discloses the synthesis of substituted 4-aminoindane derivatives via utilizing sulfonic acids for the initial dehydration of the 2-(hydroxyalkyl)-anilines and subsequent isomerization of their immediate corresponding 2-(alkenyl)-anilines towards their 4-aminoindane cyclization precursor before final and irreversible cycloisomerization towards the target compounds.

While this prior art process for producing substituted indanylamines allows the production of the desired compounds in some cases in an acceptable yield, it also exhibits disadvantages: As described, the reaction can be performed particularly well in the presence of either methanesulfonic acid (MsOH) or, most preferably, with trifluoromethanesulfonic acid (TfH) as cyclization mediator. While MsOH is a readily available bulk chemical, TfOH displays limited availability and is consequently highly expensive. Even though the majority of the acid being used can in principle be recycled, at least one equivalent forms the respective 4-aminoindane trifluoromethylsulfonate salt as an immediate product. Said equivalent and potential TfOH residues on the salt cannot be recovered via distillation and have to be neutralized by a base. The costs for raw-material consumption and wastewater treatment add up significantly to the overall process costs. This issue is inferior for the case, when MsOH is being used, due to significantly lower raw-material costs and the good biodegradability of this acid to carbon dioxide and sulfate. However, in WO 2017/133981 is reported that the application of MsOH only led to moderate yields, e. g. 52% yield by HPLC. Summarizing, the process according to WO 2017/133981 uses either a cyclization mediator which is highly expensive and which is difficult to recycle but which leads to acceptable yields or uses a cyclization mediator which is less expensive and exhibits a good biodegradability but instead leads to lower yields.

Moreover, WO 2017/133981 discloses that when certain acids other than TfOH, MsOH or polyphosphoric acid are used, no yield is obtained with this process. Especially, according to the preparation examples, no yield was generated when sulfuric acid was used as cyclization mediator at a temperature of 190° C.

With regard to the disadvantages outlined above, there is a demand for a simplified method that can be carried out industrially and economically for the general preparation of substituted 4-aminoindane derivatives. The substituted 4-aminoindane derivatives obtainable by this desired method should preferably in this case be obtained in higher yield and high purity. In particular, the desired method should enable the desired target compounds to be obtained without the need for complex purification methods such as column chromatography.

The hereinbelow-described process according to the invention achieves these objects.

The process according to the invention allows the production of substituted 4-aminoindane derivatives in a cost-efficient manner and in higher yields.

Furthermore, the process for production of substituted 4-aminoindane derivatives according to the invention allows the use of recyclable cyclization mediators during their synthesis. In particular, the process according to the invention allows the use of recyclable acids during the synthesis of said substituted 4-aminoindane derivatives. Consequently, the production of huge amounts of waste is prevented by the process according to the invention.

Furthermore, according to the invention, 4-aminoindane derivatives can be prepared by a sulfuric acid-mediated cyclization reaction. This is even more surprising since according to WO 2017/133981, no yield was obtained when sulfuric acid was used as cyclization mediator. Therefore, those skilled in the art would have expected that exposure to this acid would not lead to the production of substituted 4-aminoindane derivatives, i.e. it had been assumed that—as in the use of other Bronsted or Lewis acids—successful cyclization would not take place.

In addition it was found that 4-aminoindane derivatives can be prepared by a cyclization reaction mediated by anhydrous hydrogen fluoride (HF).

Accordingly, the present invention relates to a novel method for preparing substituted 4-aminoindane derivatives of the formula (I):

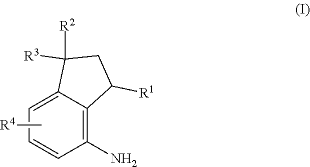

in which

R¹ represents (C₁-C₄)alkyl;

R² represents hydrogen or (C₁-C₈)alkyl;

R³ represents hydrogen or (C₁-C₈)alkyl, provided that R² and R³ are not hydrogen at the same time;

R⁴ represents hydrogen, halogen, (C₁-C₄)alkyl or (C₁-C₄)haloalkyl,

characterized in that a compound of the formula (IIa) or (IIb) or (IIc)

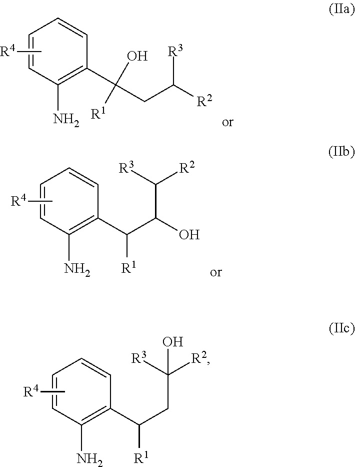

are reacted with sulfuric acid or anhydrous hydrogen fluoride (HF), wherein the definitions of the substituents R¹, R², R³ and R⁴ listed in the formulae (IIa), (IIb) and (IIc) are the same as in the formula (I).

Preferred, particularly preferred and most preferred definitions of the substituents R¹, R², R³, and R⁴ listed in the formulae (I), (IIa), (IIb) and (IIc) mentioned above are elucidated below.

It is preferable when in each case:

R¹ represents methyl or n-propyl;

R² and R³ represent methyl;

R⁴ represents hydrogen or fluorine.

It is particularly preferable when in each case:

R¹ represents methyl or n-propyl;

R² and R³ represent methyl;

R⁴ represents hydrogen.

It is most preferable when in each case:

R¹ represents n-propyl;

R² and R³ represent methyl;

R⁴ represents hydrogen.

It is also most preferable when in each case:

R¹, R² and R³ represent methyl;

R⁴ represents hydrogen.

It is also most preferable when in each case:

R¹, R² and R³ represent methyl;

R⁴ represents fluorine.

### Definitions

In the definitions of the symbols given in the above formulae, collective terms which are generally representative of the following substituents were used:

Halogen: fluorine, chlorine, bromine or iodine, preferably fluorine, chlorine or bromine, more preferably fluorine or chlorine and most preferably chlorine or bromine.

Alkyl: saturated, straight-chain or branched hydrocarbyl radical having 1 to 8, preferably 1 to 6, and more preferably 1 to 4 carbon atoms, for example (but not limited to) C₁-C₆-alkyl such as methyl, ethyl, propyl (n-propyl), 1-methylethyl (iso-propyl), butyl (n-butyl), 1-methylpropyl (sec-butyl), 2-methylpropyl (iso-butyl), 1,1-dimethylethyl (tert-butyl), pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Particularly, said group is a C1-C4-alkyl group, e.g. a methyl, ethyl, propyl, 1-methylethyl (isopropyl), butyl, 1-methylpropyl (sec-butyl), 2-methylpropyl (iso-butyl) or 1,1-dimethylethyl (tert-butyl) group.

Haloalkyl: straight-chain or branched alkyl groups having 1 to 8, preferably 1 to 6 and more preferably 1 to 4 carbon atoms (as specified above), where some or all of the hydrogen atoms in these groups are replaced by halogen atoms as specified above, for example (but not limited to) C₁-C₃-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl.

### DETAILED DESCRIPTION OF THE PROCESS

According to the invention, substituted 4-aminoindane derivatives of the formula (I) may be prepared by the reaction of an alcohol of the formula (IIa) or (IIb) or (IIc) with sulfuric acid or anhydrous hydrogen fluoride, as shown in scheme (1):

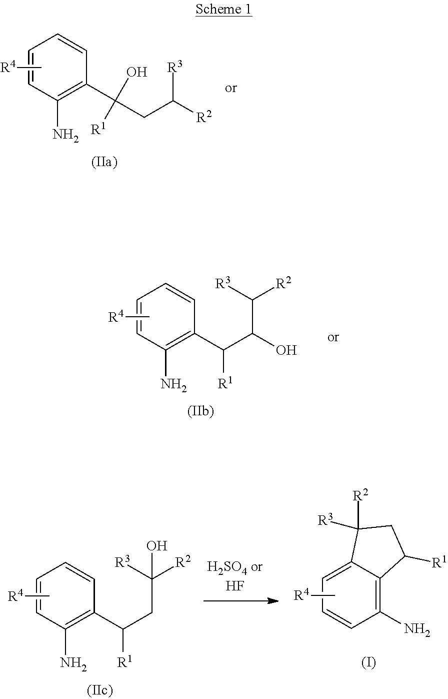

In scheme 1, the substituents R¹, R², R³ and R of the formulae (I), (IIa), (IIb) or (IIc) each have the general, preferred, particularly preferred, more preferred or most preferred meanings which have already been defined for these substituents in connection with the description of the compounds of the formulae (I), (IIa), (IIb) or (IIc).

The compounds of the formulae (IIa), (IIb) or (IIc) used as starting materials may be prepared analogously to known methods (WO 2002/38542, WO 2006/120031). Furthermore, the compound of the formula (IIa) can also be prepared by the two-fold reaction of appropriately substituted aminobenzonitriles of the formula (III) with Grignard reagents of the formulae (IVa) and (IVb) via the intermediately formed ketones of the formulae (Va) or (Vb) as shown in scheme (2).

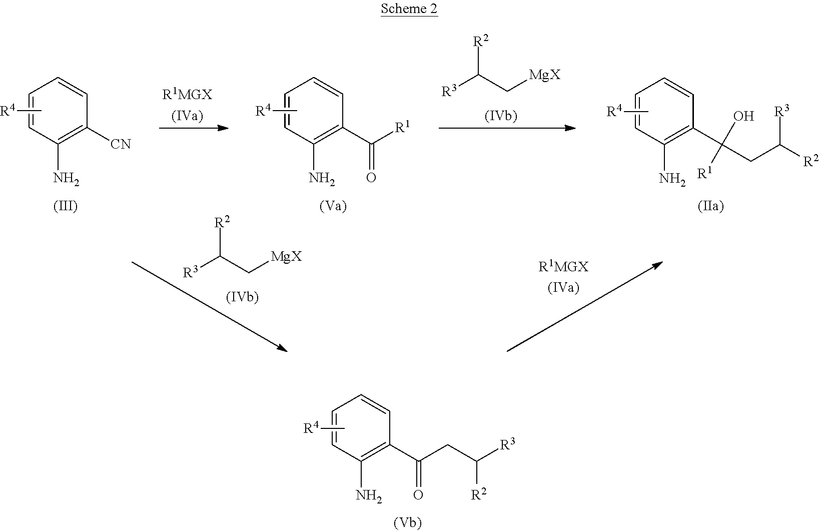

In scheme 2, the substituents R¹, R², R³ and R⁴ of the formulae (III), (IVa), (IVb), (Va), (Vb) and (IIa) each have the general, preferred, particularly preferred, more preferred or most preferred meanings which have already been defined for these substituents in connection with the description of the compounds of the formulae (I), (IIa), (IIb) or (IIc). In the formulae (IVa) and (IVb), X is preferably chlorine, bromine or iodine and particularly preferably chlorine or bromine.

The aminobenzonitriles of the formula (III) are known and in some cases commercially available.

The Grignard reagents of the formulae (IVa) and (IVb) are either commercially available or can be prepared from the corresponding chlorides, bromides or iodides by reaction with magnesium turnings by known literature methods.

To obtain the compound of the formula (I) according to the invention and as shown in scheme 1, the compound of the formula (IIa) or (IIb) or (IIc) is reacted with aqueous sulfuric acid or anhydrous hydrogen fluoride (HF), wherein the definitions of the substituents R¹, R², R³ and R⁴ of the formulae (I), (IIa), (IIb) and (IIc) each have the general, preferred, particularly preferred, more preferred or most preferred meanings which have already been defined for these substituents in connection with the above description of these compounds.

The process according to the invention is preferably carried out without a solvent or in one or more of the following solvents: ethers such as dioxane, diglyme, methyl tert-butyl ether (MTBE), tert-amyl methyl ether (TAME); nitriles such as acetonitrile (ACN) or butyronitrile; aromatic hydrocarbons such as toluene, anisole, xylenes, mesitylene; halohydrocarbons and halogenated aromatic hydrocarbons, particularly chlorohydrocarbons such as tetrachloroethylene, tetrachloroethane, dichloropropane, methylene chloride (dichloromethane, DCM), dichlorobutane, chloroform, trichloroethane, trichloroethylene, pentachloroethane, difluorobenzene, 1,2-dichloroethane, chlorobenzene, bromobenzene, dichlorobenzene, especially 1,2-dichlorobenzene, chlorotoluene, trichlorobenzene; fluorinated aliphatic and aromatic compounds such as trichlorotrifluoroethane, benzotrifluoride, 4-chlorobenzotrifluoride and water. It is also possible to use solvent mixtures.

Furthermore, the process according to the invention is particularly preferably carried out in pure aqueous sulfuric acid or anhydrous hydrogen fluoride without solvent.

Preferably, the process according to the invention is carried out at a temperature in the range of from −80° C. to 70° C., particularly preferably at a temperature in the range of from −50° C. to 30° C., more preferably at a temperature in the range of from −30° C. to 15° C.

Also preferably, if aqueous sulfuric acid is used as cyclization mediator, the process according to the invention is carried out at a temperature in the range of from 0° C. to 70° C.

Also preferably, if anhydrous hydrogen fluoride is used as cyclization mediator, the process according to the invention is carried out at a temperature in the range of from −80° C. to 20° C., particularly preferably at a temperature in the range of from −50° C. to 20° C., more preferably at a temperature in the range of from −30° C. to 20° C.

Preferably, if aqueous sulfuric acid is used as cyclization mediator, the process according to the invention is carried out at a temperature in the range of from 1° C. to 70° C.

Particularly preferably, if aqueous sulfuric acid is used as cyclization mediator, the process according to the invention is carried out at a temperature in the range of from 1° C. to 30° C.

More preferably, if aqueous sulfuric acid is used as cyclization mediator, the process according to the invention is carried out at a temperature in the range of from 1° C. to 20° C.

Even more preferably, if aqueous sulfuric acid is used as cyclization mediator, the process according to the invention is carried out at a temperature in the range of from 1° C. to 15° C.

Most preferably, if aqueous sulfuric acid is used as cyclization mediator, the process according to the invention is carried out at a temperature in the range of from 5° C. to 15° C.

The amount of the employed cyclization mediator may be varied over a wide range but is preferably in the range of from 3-45 molar equivalents, preferably of from 6 to 40 molar equivalents, especially preferably of from 9 to 35 molar equivalents based on the total amount of the compound of the formula (IIa) or (IIb) or (IIc).

If aqueous sulfuric acid is used as cyclization mediator, its used amount may be varied over a wide range but is preferably in the range of from 3-18 molar equivalents, preferably of from 6 to 15 molar equivalents, especially preferably of from 9 to 12 molar equivalents based on the total amount of the compound of the formula (IIa) or (IIb) or (IIc).

If anhydrous hydrogen fluoride is used as cyclization mediator, its used amount may be varied over a wide range but is preferably in the range of from 15-45 molar equivalents, preferably of from 20-40 molar equivalents, especially preferably of from 25-35 molar equivalents based on the total amount of the compound of the formula (IIa) or (lIb) or (IIc).

The process according to the invention is generally conducted at standard pressure but may be carried out either under reduced pressure or at elevated pressure—generally between 0.1 and 100 bar.

Preferably, when HF is used as the cyclization mediator in the process according to the invention, HF is used in anhydrous form, optionally as solution in organic solvents, more preferably HF is used in anhydrous form with a boiling point of 20° C. (i.e. without any organic solvents and free of water).

Depending on the type of substituents, the compound of the formula (I) can occur as geometric and/or optical isomers or as their corresponding isomeric mixtures in various compositions. These isomers are, for example, enantiomers, diastereomers or geometric isomers. As a consequence, the invention described herein includes both the pure stereoisomers and every mixture of these isomers.

The desired compound of the formula (I) can be isolated and purified by diluting the reaction mixture with water with subsequent crystallization and release of the free 4-aminoindane derivative. Such methods are known to those skilled in the art and particularly include the crystallization of the 4-aminoindane derivative ammonium salt from water and liberation of the free 4-aminoindane derivative via neutralization and extraction with an organic solvent.

The present invention is elucidated in detail by the examples which follow, although the examples should not be interpreted in such a manner that they restrict the invention.

### PREPARATION EXAMPLES

### Example (a): Preparation of rac-1,1-dimethyl-3-propyl-indan-4-amine

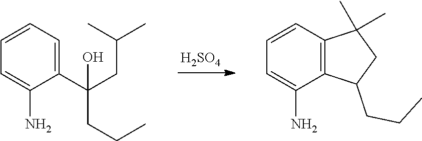

In a 25 mL three-necked reaction flask equipped with a thermometer was placed 15.4 mL of concentrated sulfuric acid (97% purity). To the acid was added 6.43 g (97% purity, 28.18 mmol, 1.0 eq) of rac-4-(2-aminophenyl)-2-methyl-heptan-4-ol dropwise at 15° C. internal temperature. After the first dissolution of the substrate a jelly-like solid separates, which then slowly dissolves again. The solution was allowed to reach 22° C. and was stirred for 8 hours at this temperature until full conversion was obtained according to HPLC analysis. The solution was then added to 80 mL of deionized water. A white solid precipitated, which was filtered off. The wet solid was then suspended in 50 mL of deionized water and sodium hydroxide was used to adjust pH 7. The solid transformed into an oily layer, which was extracted with 50 mL of ethyl acetate. After phase separation, the organic phase was washed with 50 mL of saturated brine and dried over magnesium sulfate. After filtration of the drying agent, the organic phase was concentrated via distillation at 40° C. down to a vacuum of 5 mbar to leave 5.37 g (81% purity, 21.3 mmol, 75% yield) of rac-1,1-dimethyl-3-propyl-indan-4-amine as a dark red oil. ¹H-NMR (600 MHz; CDCl₃)=7.02 (t, J=7.5 Hz, 1H), 6.59 (d, J=7.5 Hz, 1H), 6.47 (d, J=7.5 Hz, 1H), 3.56 (bs, 2H), 3.11-3.06 (m, 1H), 2.09 (dd, J=12.0 Hz, 24.0 Hz, 1H), 1.92-1.86 (m, 2H), 1.76 (dd, J=6.0 Hz, 12.0 Hz, 1H), 1.55-1.32 (m, 2H), 1.30 (s, 3H), 1.21 (s, 3H), 0.97 (t, J=8.0 Hz, 3H).

### Example (b) Preparation of rac-1,1-dimethyl-3-propyl-indan-4-amine (Comparative Example)

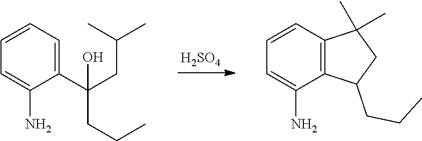

In a 8 mL screw-capped vial was placed 0.5 g (87% purity, 2.06 mmol, 1.0 eq) of rac-4-(2-aminophenyl)-2-methyl-heptan-4-ol and heated to the temperature, which is indicated in the table. To the starting material was added 2.0 g (97% purity, 19.7 mmol, 9.6 eq) of concentrated sulfuric acid. The reaction was mixed at the indicated temperature until full conversion of starting material was observed via HPLC monitoring. The yield was determined via quantitative ¹H-NMR spectroscopy of the reaction mixture using dibromomethane as internal standard.

| No. | Temperature/° C. | Time/h | Yield |
|---|---|---|---|
| 1 | 30 | 8 | 71% |
| 2 | 50 | 6 | 65% |
| 3 | 70 | 6 | 61% |
| 4 | 90 | 6 | 52% |
| 5 | 120 | 4 | 41% |
| 6 | 140 | 4 | 40% |
| 7 | 180 | 4 | 28% |

¹H-NMR (600 MHz; CDCl₃) δ=7.02 (t, J=7.5 Hz, 1H), 6.59 (d, J=7.5 Hz, 1H), 6.47 (d, J=7.5 Hz, 1H), 3.56 (bs, 2H), 3.11-3.06 (m, 1H), 2.09 (dd, J=12.0 Hz, 24.0 Hz, 1H), 1.92-1.86 (m, 2H), 1.76 (dd, J=6.0 Hz, 12.0 Hz, 1H), 1.55-1.32 (m, 2H), 1.30 (s, 3H), 1.21 (s, 3H), 0.97 (t, J=8.0 Hz, 3H).

### Example (c): Preparation of rac-1,1-dimethyl-3-propyl-indan-4-amine Using Anhydrous HF

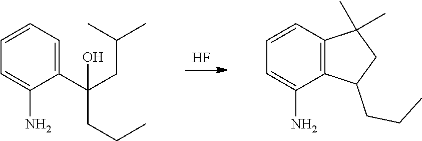

To a 20 mL Nalgene® laboratory bottle charged with 0.5 g of 4-(2-amino-phenyl)-2-methyl-heptan-4-ol (87.1% purity, 2.25 mmol) at −30° C. was added 1 g (1 mL, 50 mmol, 22 eq) of precooled (+5° C.) anhydrous hydrogen fluoride (b.p. 19.5° C., m.p −83.6° C.). The bottle was sealed via stopper, the reaction mixture was allowed to warm to room temperature (25° C.) and stirred at this conditions for 24 hrs.

After the reaction was complete, the bottle was cooled down to +5° C. and opened. The excess of hydrogen fluoride was allowed to evaporate at open air under the fume hood. The oily residue was then treated with 10% aqueous solution of NaHCO₃ (10 mL) until a pH value of 7 was obtained (CO₂ evolution occurred) and extracted with CH₂Cl₂ (2×10 mL). The combined dichloromethane extracts were washed with H₂O (10 mL), dried over Na₂SO₄ and evaporated under reduced pressure to leave 0.46 g (68% purity, 1.62 mmol, 72% yield) of rac-1,1-dimethyl-3-propyl-indan-4-amine as a yellow oil.

NMR (400 MHz; CDCl₃)=7.05 (t, J=8.0 Hz, 1H), 6.62 (d, J=8.0 Hz, 1H), 6.51 (d, J=8.0 Hz, 1H), 3.62 (bs, 2H), 3.07-3.02 (m, 1H), 2.12 (dd, J=12.0 Hz, 24.0 Hz, 1H), 1.91-1.80 (m, 2H), 1.78 (dd, J=6.0 Hz, 12.0 Hz, 1H), 1.56-1.34 (m, 2H), 1.33 (s, 3H), 1.24 (s, 3H), 0.99 (t, J=8.0 Hz, 3H).

1. A process for preparation of a compound of formula (I) 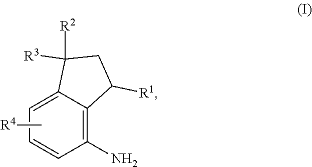
wherein
R¹ represents (C₁-C₄)alkyl;
R² represents hydrogen or (C₁-C₈)alkyl;
R³ represents hydrogen or (C₁-C₈)alkyl, provided that R² and R³ are not hydrogen at the same time;
R⁴ represents hydrogen, halogen, (C₁-C₄)alkyl or (C₁-C₄)haloalkyl,
comprising reacting a compound of formula (IIa) or (IIb) or (IIc) 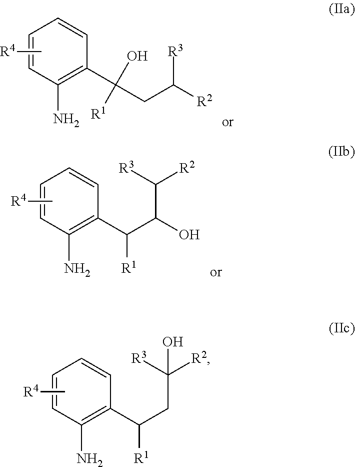
with aqueous sulfuric acid or anhydrous hydrogen fluoride at a temperature in a range of from of −80° C. to 70° C.

2. The process according to claim 1, wherein R¹ is n-propyl, R² and R³ each are methyl and R⁴ is hydrogen.

3. The process according to claim 1, wherein R¹, R² and R³ are methyl and R⁴ is hydrogen.

4. The process according to claim 1, wherein the process is carried out at a temperature in a range of from 1° C. to 30° C., optionally at a temperature in a range of from 1° C. to 20° C.; optionally at a temperature in a range of from 1° C. to 15° C.

5. The process according to claim 1, wherein the process is carried out at a temperature in a range of from 5° C. to 15° C.

6. The process according to claim 1, wherein an aqueous sulfuric acid having a concentration of at least 85w % is used.

7. The process according to claim 1, wherein an aqueous sulfuric acid is used that has a concentration in a range of from 85w % to 97w %, optionally that has a concentration in a range of from 88 w % to 92 w %, optionally the concentration of the aqueous sulfuric acid is 90 w %.

8. The process according to claim 1, wherein the amount of used aqueous sulfuric acid or anhydrous hydrogen fluoride is in a range of from 3-45 molar equivalents, optionally of from 6 to 40 molar equivalents, optionally of from 9 to 35 molar equivalents based on a total amount of the compound of formula (IIa) or (Ib) or (IIc).

9. The process according to claim 1, wherein the reaction can be conducted in the presence or absence of a solvent, optionally the reaction is conducted in absence of a solvent.

10. Process for preparation of a compound of formula (V) 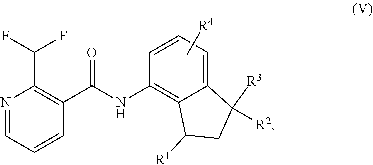
comprising the process of claim 1, and further comprising reacting the compound of formula (I) with a compound of formula (VI). 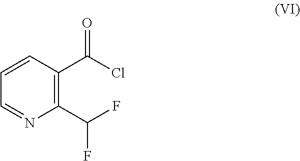 [SMILES: O=C(Cl)c1cccnc1C(F)F]
to obtain the compound of formula (V).
